(19) United States
(12) Patent Application Publication
(10) Pub. No.: US 2006/0013354 A1
(43) Pub. Date: Jan. 19, 2006

(54) Method for reconstructing tomograms from detector measured data of a tomography unit

(76) Inventor: Bjoern Heismann, Erlangen (DE)

Correspondence Address: HARNESS, DICKEY & PIERCE, P.L.C., P.O.BOX 8910, RESTON, VA, 20195, US

(21) Appl. No.: 11/181,734

(22) Filed: Jul. 15, 2005

(30) Foreign Application Priority Data
Jul. 16, 2004 (DE) 10 2004 034 502.3

(51) Int. Cl. H05G 1/60; A61B 6/00; G01N 23/00; G21K 1/12

(52) U.S. Cl. 378/4

(57) ABSTRACT

A reconstruction method is used in computed tomography, wherein an examination object is scanned with a beam that rotates during scanning about a system axis. In the method, firstly the absorption coefficients of subvolumes relatively remote from the system axis are determined. Subsequently the absorption coefficients of the subvolumes situated relatively nearer to the system axis are determined iteratively, taking account of the absorption coefficients already calculated.

The present application hereby claims priority under 35 U.S.C. §119 on German patent application number DE 10 2004 034 502.3 filed Jul. 16, 2004, the entire contents of which is hereby incorporated herein by reference.

### FIELD

The invention generally relates to a method for reconstructing tomographs. For example, it may relate to a method for reconstructing them from detector measured data of a tomography unit having at least one radiation source that is moved about the system axis, and at least one oppositely situated, at least single row detector that measures the absorption of the radiation, emanating from the radiation source, after the penetration of an examination object, at least the radiation source revolving on an imaginary cylindrical surface of the examination object and in the process scanning by a beam, this examination object, which lies in a scanning volume formed by the rays.

Moreover, the invention generally relates to a CT unit that is equipped with a device/method for carrying out reconstructions of CT images.

### BACKGROUND

Two different methods are generally known in principle with reference to the reconstruction of CT images. Reference is made in this regard to the publication entitled “Computertomographie [Computed tomography] Willi A. Kalender, ISBN 3-89578-082-0”. The two variants of the methods of calculation are presented in chapter 1.2.3. These are, on the one hand, an explicit method of calculation in which a cross section of the examination object is divided into N×N matrix elements, and these N² unknown values of the N×N image matrix are determined by solving a system of linear equations.

In the simplest case of a 2×2 image matrix of only four pixels, two measurements each from two directions result in a system of four equations with four unknowns that can easily be solved. With a higher resolution, and therefore with an enlarged matrix, there is a disproportionately sharp rise in the computing times, and so it is scarcely possible to carry out the method in practice for a matrix of the current order of magnitude of 512×512 pixels. Furthermore, this also gives rise in principle to error propagation problems that lead to insolubility of such systems of linear equations, and thus excludes explicit calculation in practice.

Instead of the explicit calculation of the image values, use is made at present in practice of an approximation method in which the image is produced from the measured sinograms by convolution and back projection. Particularly in spiral CT, this method is further supplemented by a preceding rebinning, that is to say a reordering of the scanning rays, if appropriate in tandem with interpolation methods that use the measured data obtained to produce a measured data sequence in the respectively desired geometric form, CT images subsequently being reconstructed by convolution and back projection. These reconstruction methods are also very complicated in part and require enormous computing capacities despite approximate calculation.

### SUMMARY

The inventor, in at least one embodiment, has assumed the task of finding a method for reconstructing tomograms from detector measured data of a tomography unit that, in a fashion better than the method previously known, examines the actual geometry of the scanning systems of modern CT units, in the case of which radiation sources emit a fan-shaped beam onto a detector, and this radiation source revolves around the considered examination object on an—imaginary—cylindrical surface of the examination object.

The inventor has realized that the calculation of the absorption coefficients of subvolumes of an object can be carried out with particular ease when the examination region is subdivided into a multiplicity of concentrically arranged shells that are subdivided per se once again into individual shell segments, the absorption coefficients of the individual shell segments being determined progressively from the outer shell down to the inner shell consecutively. Starting from an edge ray of the fan of rays, in this case during the revolution of the radiation source around the examination object, the variation in the intensity of the beam in relation to the shell segments penetrated and to the path lengths of the ray through these segments considered is set, as a result of which it is possible to determine the absorption coefficients of the shell segments of the outer shell with the aid of simple computing methods and a low computational outlay.

If these absorption coefficients are known, a ray lying further inside can be considered. The absorption coefficients of the outer shell segments already known on the beam path are now taken into account when calculating the absorption coefficients of the shell lying further inside such that the mathematical task is not substantially enlarged inward by a further iteration, and that it is possible in this way, overall, to determine all the shells and their shell segments iteratively with reference to their absorption coefficient progressively from the outside to the inside and without a higher computational outlay with the aid of linear equations that are easy to solve.

An advantage of at least one embodiment of this outlined method resides in the fact that the geometric subdivision of the examination object is a very close approximation of the actual method of examination, since there is no problem in also being able to assign a specific shell of the examination region to each individual ray that, starting from the radiation source, reaches a detector element in the detector. This facilitates the proposed method of calculation enormously.

Approximately 3×10⁶ memory locations and approximately 10⁹ operations of addition and multiplication are required for calculation by a disk, corresponding to a CT tomogram, that is divided into 700 concentrically arranged shells that correspond to the number of the detector elements of a detector row, each shell being subdivided into 1000 shell segments. This can be carried out in a reasonable time with the aid of modern central processors. If use is made for this purpose of parallel computing structures such as, for example, FPGA (Floating Point Grid Array) processors, the computing time is lowered drastically. This is so, in particular, because there is no problem in calculating the absorption coefficients of all the shell segments of a shell in parallel.

However, this basic idea can be applied not only given a shell-like division of the scanning volume, but can also be generalized to any desired divisions as long as in the iterative process subvolumes lying outside are firstly considered and their absorption coefficients are calculated with the aid of the absorption values of rays lying outside, and subsequently recalculation is continued in a stepwise fashion toward the center or toward the system axis, the then already known absorption coefficients of outer subvolumes that are likewise penetrated by the rays lying further inside featuring in the calculation in each case.

The inventor proposes, in accordance with at least one embodiment of this basic idea, a reconstruction method in computed tomography in the case of which an examination object is scanned with a beam that rotates during scanning about a system axis, wherein firstly the absorption coefficients of subvolumes that are most remote from the system axis are determined, and subsequently the absorption coefficients of the subvolumes situated close to the system axis are determined iteratively taking account of the absorption coefficients already calculated.

At least one embodiment of this inventive method can advantageously be carried out in each case for an individual slice plane or for a number of individual slice planes independently of one another.

Moreover, at least one embodiment of the inventive method can be used in conjunction with spiral scanning and for complete scanning volumes of at least substantially cylindrical design by firstly calculating subvolumes at the edge using rays at the edge and subsequently iteratively calculating subvolumes, lying further inside and approaching one another in relation to the system axis, with reference to their absorption coefficients, doing so in each case by taking account of the already calculated absorption coefficients lying further outside.

At least one embodiment of the method can be used advantageously in this way with multirow detectors and/or multifocal and/or multitube systems.

In a more concrete refinement of at least one embodiment, the inventor furthermore proposes to improve the method known per se for reconstructing tomograms from measured data of a tomography unit in the case of which at least one radiation source is moved about a system axis and at least one oppositely situated, at least single row, detector is provided that measures the absorption of the radiation, emanating from the radiation source, after the penetration of an examination object, wherein at least the radiation source preferably also the detector, revolves around the examination object on an imaginary cylindrical surface, preferably on a multiplicity of circular tracks or a spiral track, and in the process scans this examination object, which lies in a scanning volume formed by the rays, with a beam.

The scanning volume is divided into a multiplicity of subvolumes, and use is made initially in the beam of a ray, preferably an edge ray of the detector, that is remote from the system axis and whose absorption is used to determine the absorption coefficients of the subvolumes penetrated by this ray. Subsequently, iterative use is made of rays closer to the system axis and of their absorption in order to determine the as yet unknown absorption coefficients of as yet unconsidered subvolumes taking account of already known absorption coefficients of already calculated subvolumes.

It may be pointed out that the beginning of iteration must occur at a ray that admittedly need not correspond necessarily to the edge ray of a detector, but must lie so far outside that this is the first ray counted from outside that intersects the examination object. Rays that do not intersect the examination object need not be taken into account during the iterative calculation, but also do not interfere in the iteration process, since they merely supply absorption values of air in a uniform fashion.

From a consideration of a geometrically particularly favorable division of the scanning volume, the method in accordance with at least one embodiment resides in that the scanning volume is divided into a multiplicity of concentric shells, and the shells are subdivided in turn into shell segments, in which case firstly the absorption coefficients of the outer segments are determined. Thereupon, the absorption coefficients of the shell segments lying further inside, are determined iteratively taking account of the absorption coefficients, calculated meanwhile of shell segments lying further outside, until the center of the scanning volume is reached.

This iterative method, in which the absorption coefficients of individual shell segments of concentrically arranged shells are determined progressively from outside to inside in an interactive fashion, has the advantage that the computing time required therefor turns out to be very short.

One advantageous refinement of an embodiment of this method can reside in that the shells are divided into individual shell segments of identical thickness in the radial direction. It is also possible for the shell segments of the shells to have the same length in the circumferential direction.

As an alternative to constructing shell segments of the same length, the shell segments can also be fashioned such that their length corresponds to a specific segment angle, as a result of which the outer shells have a relatively large length, and the shells situated most centrally have minimum extension of length.

In accordance with a further variant refinement of at least one embodiment, the shell segments can also be constructed such that they all have the same cross-sectional surface, measured perpendicular to the system axis.

The method according to at least one embodiment of the invention is particularly advantageous when, proceeding from the radiation source, as a rule from one or more foci to a specific detector element, each ray is assigned a shell at a constant distance from the system axis. In this case, the geometric arrangement of the detector and of the radiation source should be selected with particular advantage such that each connecting line between the location of origin of the ray and a detector element of a row has a differing distance from the central system axis. It is possible in this way to avoid redundancies and optimize the resolution.

The method can advantageously also be designed such that the shell segments have an imaginary centroid line which runs about the examination object in accordance with the spatial path from the outlet of the radiation to the respective detector element of the detector such that, in each case a ray is tangential to this centroid line on its path during the movement of focus and detector. In this case, the ray segments have an imaginary centroid line whose course corresponds to the course of the feet of the perpendicular from the system axis to the respective ray. This configuration of the ray segments has the result that the measured absorption coefficients of a segment correspond in a largely optimum way to the mean value of the absorption coefficient of this segment. It is to be pointed out in this context that the absorption coefficients can certainly differ over the volume of a shell segment, but it is assumed in an idealized way in the present invention that each shell segment is associated with a mean absorption coefficient.

It can also be advantageous when each shell segment has an imaginary centroid line that corresponds to a segment of concentrically arranged circles and helical lines about the system axis. Such an arrangement is particularly advantageous when the scanning takes place by means of a so-called spiral CT in which the radiation source is guided on a spiral path about the examination object.

In a concrete design of the method, the absorption coefficients \vec{μ}ₛ of the shell segments sₛᵢ of the shell Sₛ can be calculated iteratively using the following formula

$${\vec{μ}}_{s} = {L}_{s}^{- 1} ( {\vec{A}}_{s} - {\sum }_{i = 1}^{s - 1} {L}_{s , i} {\vec{μ}}_{i} ) ,$$

Lₛ being the path length matrix of the defining ray of the shell Sₛ, L_{s,i} being the path length matrices of the rays i=1 . . . s lying further outside with reference to the system axis, and Aₛ being the partial sinogram belonging to the shell Sₛ—see below for a more precise definition.

The path length matrix Lₛ or L_{s,i} includes the intersection lengths of a ray Rₛ with the segments of the shell Sₛ (for Lₛ) or of the shells i=1 . . . s (for L_{s, i}) lying further outside. The absorption law can therefore be written for each ray Rₛ as
\vec{A}ₛ=L_{s,1}\vec{μ}₁+ . . . +L_{s,s-1}\vec{μ}ₛ₋₁+Lₛ\vec{μ}ₛ.
Lₛ and L_{s,i} have a particularly advantageous, permutative structure for the rotationally symmetrical selection of the shell segments: the first row already includes the entire information relating to the path lengths, here termed \vec{l}ᵣₒₜ. All further rows are permutations of this first row. The matrix Lₛ can then be assembled as: Lₛ=[\vec{l}ᵣₒₜ, \vec{l}ᵣₒₜ permuted by one position, \vec{l}ᵣₒₜ permuted by 2 positions, . . . \vec{l}ᵣₒₜ permuted by s−1 positions].

An example would be \vec{l}ᵣₒₜ =[0.2, 0.4, 0.7, 0]. \vec{l}ᵣₒₜ is then permuted by 1 position=[0, 0.2 0.4, 0.7] and so on, and therefore

$${L}_{s} = ( \begin{matrix}0.2 & 0.4` & 0.7 & 0 \\ 0 & 0.2 & 0.4 & 0.7 \\ 0.7 & 0 & 0.2 & 0.4 \\ 0.4 & 0.7 & 0 & 0.2\end{matrix} )$$

In this case, the totality of the measured values of all the detector channels 1 . . . n over the projections 1 . . . p is denoted as sinogram A. A is a matrix having the dimensions [1 . . . n, 1 . . . p].

The partial sinogram Aₛ corresponds to the measured data of the sth detector channel of the sinogram A. Aₛ is a vector with the dimension [1 . . . p].

It may be expressly remarked that, in addition to the preferred division of the scanning volume into shells and shell segments, other divisions into subvolumes with arbitrary geometrical shapes are also possible although no beam-shaped volumes are possible that extend from the center to the edge regions. Rectangular or square or else hexagonal shapes may be named, by way of example; it is advantageous but not mandatory, in this case, when the subvolumes fill up the entire scanning volume without interspaces.

In the case of the previously described methods according to an embodiment of the invention, it is assumed in an idealized way that the absorption coefficient remains independent of the spectral variation of the ray upon passage through the examination object. It is known that is an idealized assumption, because X-rays, in particular, vary their energy spectrum when passing through an examination object, the result being a progressive hardening of the energy spectrum with increasing path length of the ray through the examination object.

Modern CT reconstruction algorithms, which for the most part are based on FFT back-projection methods, are also based on the approximation assumption that a line integral of the absorption coefficient μ_{eff}(\vec{r}) follows from the air measurement I₀ and the measurement I

$$1 n \frac{I}{{I}_{0}} = {\int }_{( L )}^{} {μ}_{eff} ( \vec{r} ) ⅆ \vec{r} .$$

The energy weighting of the absorption coefficient is neglected in this case because an exact formulation then runs

$$1 n \frac{I}{{I}_{0}} = 1 n \frac{{\int }_{E}^{} S ( E ) D ( E ) {ⅇ}^{{\int }_{( L )}^{} κ ( E , r ) ⅆ r} ⅆ E}{{\int }_{E}^{} S ( E ) D ( E ) ⅆ E} = 1 n {\int }_{E}^{} w ( E ) {ⅇ}^{- {\int }_{( L )}^{} κ ( E , r ) ⅆ r} ⅆ E$$

S(E) and D(E) are in this case the input tube spectrum and the detector absorption probability. The energy weighting w(E) is defined in accordance with

$$w ( E ) = \frac{S ( E ) D ( E )}{\int S ( E ) D ( E ) ⅆ E} .$$

κ(E,r) is the differential absorption coefficient for the energy E and at the location r.

Only when the 2nd equation is approximated for small distances or small objects is the first-named approximation

$$1 n \frac{I}{{I}_{0}} {\approx }^{Linearization} - {\int }_{( L )}^{} {\int }_{E}^{} w ( E ) κ ( E , \vec{r} ) ⅆ E ⅆ \vec{r} = - {\int }_{E}^{} {μ}_{eff} ( \vec{r} ) ⅆ \vec{r}$$

where

$${μ}_{eff} ( \vec{r} ) \approx {\int }_{E}^{} w ( E ) κ ( E , \vec{r} ) ⅆ E$$

obtained again.

This approximation has a few direct consequences in practice:

The output spectrum of the tube S(E) has experienced a hardening after it passes through highly absorbent materials such as bones in humans, that is to say the energy centroid has been displaced to higher energies. The approximation equation for small and weakly absorbing objects is attended by a substantial error for the corresponding projections. This results in typical CT artifacts such as the “bone shadow” in FIG. 1, for example. In some circumstances, these can greatly impair the interpretation of the area affected. Even in the case of measurements of soft part tissues, the radiation hardening leads as error to a “dish shape”, that is to say the HU values are depressed toward the center. Without hardening correction, the result in a 40 cm water phantom is, for example, a depression by approximately 20 HU at the center.

Spectral CT methods function exactly only in conjunction with an accurate solution.

Consequently, in an additional improved variant of at least one embodiment of the method, the inventor proposes that during the iterative measurement of the absorption coefficients their energy dependence is taken into account. On the one hand, this can be done by way of model assumptions, but on the other hand the energy-dependent variation in intensity of the radiation can also be measured after passage through the examination object.

The actual measurement of the spectral variation upon passage of the ray through an examination object can be done, for example, in that the total variation in intensity of at least two rays having a known and different energy spectrum on the same beam path is measured upon passage through the examination object and the spectral dependence of the absorption coefficient is deduced therefrom per shell segment.

This can preferably be performed in that use is made of two radiation sources with different spectra and in each case an oppositely situated detector that are preferably arranged in such a way that they revolve on a congruent track during the scanning of the examination object.

On the other hand, there is the possibility of scanning the examination object with a beam of known energy spectrum and of individually measuring the changed energy spectrum of each ray after passage through the examination object. The knowledge of the individual variation of the energy spectrum of the ray can then be used to deduce the energy dependence of the absorption coefficients of the individual shell segments by computation.

The energy spectrum of the rays can preferably be divided into two or three mean energies such that the absorption coefficients are calculated with reference to these two or three mean energies.

An intensity value of a primary color is assigned per energy to the value of the energy-dependent absorption coefficients when displaying the CT tomograms, a color display of the CT image in conjunction with color shaping as a function of the magnitude of the energy-dependent absorption coefficient resulting therefrom. It is possible for this purpose to make use, for example, of red-green-blue or yellow-magenta-cyan in additive color mixing, or correspondingly of other primary colors in subtractive color mixing.

This method is particularly advantageous when the absorption coefficients are measured with reference to two or three mean energies such that a simple assignment of a specific energy is undertaken to a specific primary color or the complementary color thereof.

The following advantages are to be expected for the newly proposed reconstruction method.

Physically correct reconstruction: tube and detector characteristics are likewise taken into account, like beam hardening effects. The image quality is thereby significantly enhanced in high absorption areas.

Natural coordinate selection and rapid selection of the image section and of the sharpness-to-noise ratio: at present, the core of the reconstruction fixes the sharpness/resolution impression of the image. The field of view (FOV) is determined before the measurement. At least one embodiment of the present method, by contrast, supplies a result that is firstly independent of the display and has a maximum information content of the measurement. This is subsequently imaged in an area, that can be selected by the user, with adjustable sharpness/resolution. Changes in the resolution in favor of the noise and vice versa, as well as subsequent changes in FOV of the user are possible without a problem and rapidly, and this substantially improves the dose usage and image evaluation by the physician.

Adapted filtering direction: the information generated by the algorithm can be filtered in an optimum way, since the coordinates of the method correspond to the discretization of the detector aperture and to the aperture/convolution, on the other hand.

Detector implementability: the method can also be implemented rationally on a part of the system co-rotating with the detector owing to the imaging on to a maximum information content.

Exact determination of the absorption coefficient and starting point for quantitative and spectral methods: the algorithm can be combined with quantitative methods or contrast-enhancing methods.

Selectable weighting functions: the effective absorption coefficient μ_{eff} can be determined with the aid of a constant and known energy weighting w(E). Optimizations to new contrasts are possible in conjunction with spectral CT measurements by creating an application-specific wₒₚₜ(E)

Application for two-dimensional detectors: the proposed algorithm is not limited by geometric approximations. It is therefore also suitable for any desired fan angle and 4D volume reconstruction. The 3D extension of the algorithm can be fully parallelized with reference to the row evaluation, that is to say the method can also in particular be advantageously implemented for a multi-computer or parallel computer system. In the final analysis, it is also possible to correct the Heel effect of the tube, the spectral composition of variation, which varies in the z-direction, as an unconsidered problem of relatively large fan angles.

Optimum dose usage: in a fashion coupled with the previous point, the algorithm offers a theoretically complete dose usage, since the reconstruction geometry permits the use of any X-ray striking the patient for the purpose of data reconstruction.

Ring artifact correction: a ring artifact correction (balancing) can be implemented very easily by selecting the reconstruction coordinate.

Scattered ray correction: information relating to the generation of scattered rays is obtained during the reconstruction procedure. This can advantageously be used for re-iteration in order to reduce scattered rays.

Additional features and advantages of the invention emerge from the following description of example embodiments with reference to the drawings.

### BRIEF DESCRIPTION OF THE DRAWINGS

The invention is described below in more detail using example embodiments and with the aid of the figures, in which case it may be pointed out that only the elements needed for an immediate understanding of the invention are shown. The following reference symbols are used in this case: 1: computed tomography unit; 2: X-ray tube; 3: detector; 4: patient couch; 5: system axis; 6: gantry; 7: patient; 8: memory; 9: data/control line; 10: central processor; 11: screen; 12: keyboard; 13: focus; 14: section; 15: scanning volume; 16: rectangular subvolume; 17: hexagonal subvolume; A: sinogram; Aₛ: partial sinogram; D: detector; Dₛ: detector elements; lₛ: passage lengths of the rays through shell segments; Lₛᵢ: matrices; Rₛ: X-ray beams; Sₛ: shells; sₛᵢ: shell segments; φ: angle of rotation of the X-ray tubes; μₛᵢ: absorption coefficients.

The drawing shows in detail:

FIG. 1: a CT unit with central processor;

FIG. 2: a section through the beam path with shell-type division of the scanning volume;

FIG. 3: a sinogram;

FIG. 4: a partial sinogram;

FIG. 5: an illustration from FIG. 2 plus two angularly offset ray courses;

FIG. 6: a section from FIG. 5;

FIG. 7: checkerboard-type subvolumes;

FIG. 8: subvolumes arranged in the form of honeycombs; and

FIG. 9: a 3D illustration of a shell-type division of the scanning volume in the case of multirow detectors.

### DETAILED DESCRIPTION OF THE EXAMPLE EMBODIMENTS

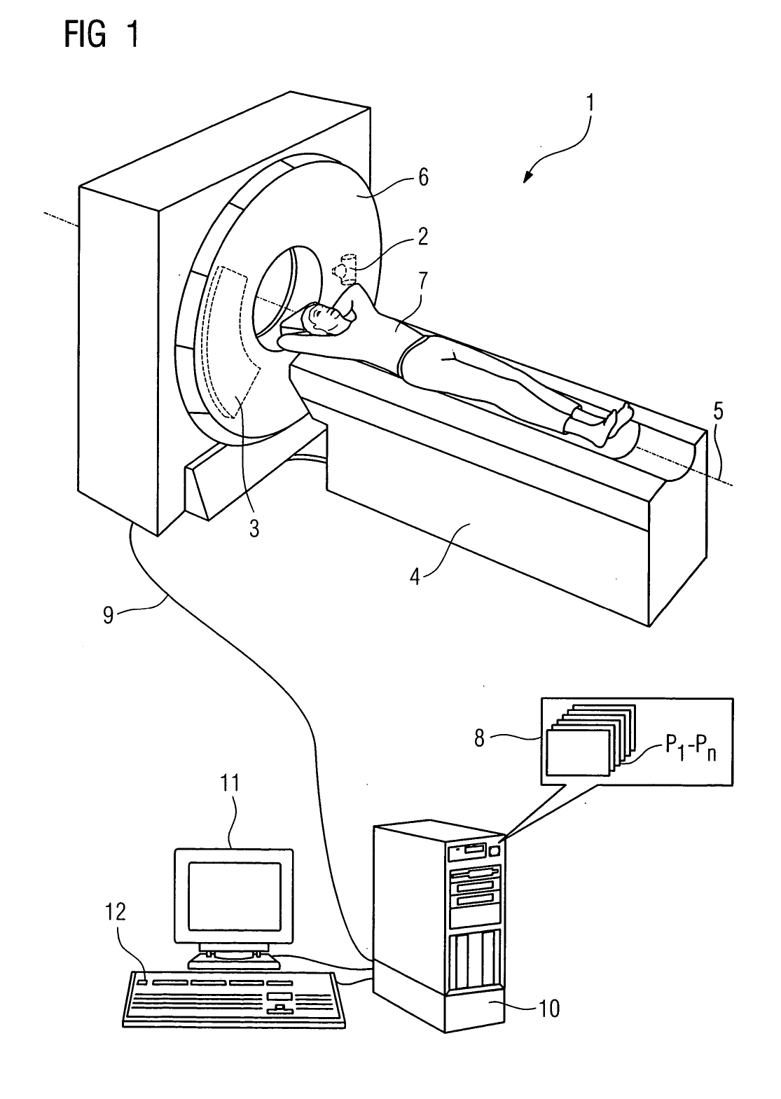

A computed tomography unit 1, known per se—as illustrated in FIG. 1—can be used to execute the method according to at least one embodiment of the invention. Such a computed tomography unit 1 has at least one X-ray tube 2 with at least one focus that generates a beam that strikes an oppositely situated detector 3.

In the design of the computed tomography unit shown here, the X-ray tube 2 with the detector 3 moves on a gantry 6 about an examination object—here a patient 7—in circular fashion and in so doing scans the examination object with its X-rays. The absorption of the X-rays is measured in the detector 3 by means of a multiplicity of detector elements, directed via a data and control line 9 to a central processor 10 and stored and processed there.

In order to operate the central processor 10, and thus also the computed tomography unit 1, the central processor has a screen 11 and an input unit in the form of a keyboard 12, which can be used both to control and to output the computed tomography pictures produced. The actual calculation method takes place in the central processor 10, which has a memory—illustrated here symbolically by reference symbol 8, in which the sequential programs P₁-Pₙ are also stored in addition to the data.

In accordance with an embodiment of the inventive method, the examination object 7 can be scanned spirally along the system axis 5 by continuously feeding the patient couch 4. A simpler variant consists in carrying out the feed sequentially such that a feed takes place after each 360° scan, and the actual scanning is undertaken with the examination object 7 at rest. Both variants can be carried out using the method according to at least one embodiment of the invention.

It may also be pointed out that it is possible to use both single-row and multirow detectors. One or more X-ray tubes each having one or more foci with, in turn, a single or a number of moving or stationary detectors can be used. All that is essential to the method is that a beam of fan-shaped design scans the examination object in a rotary movement about the system axis.

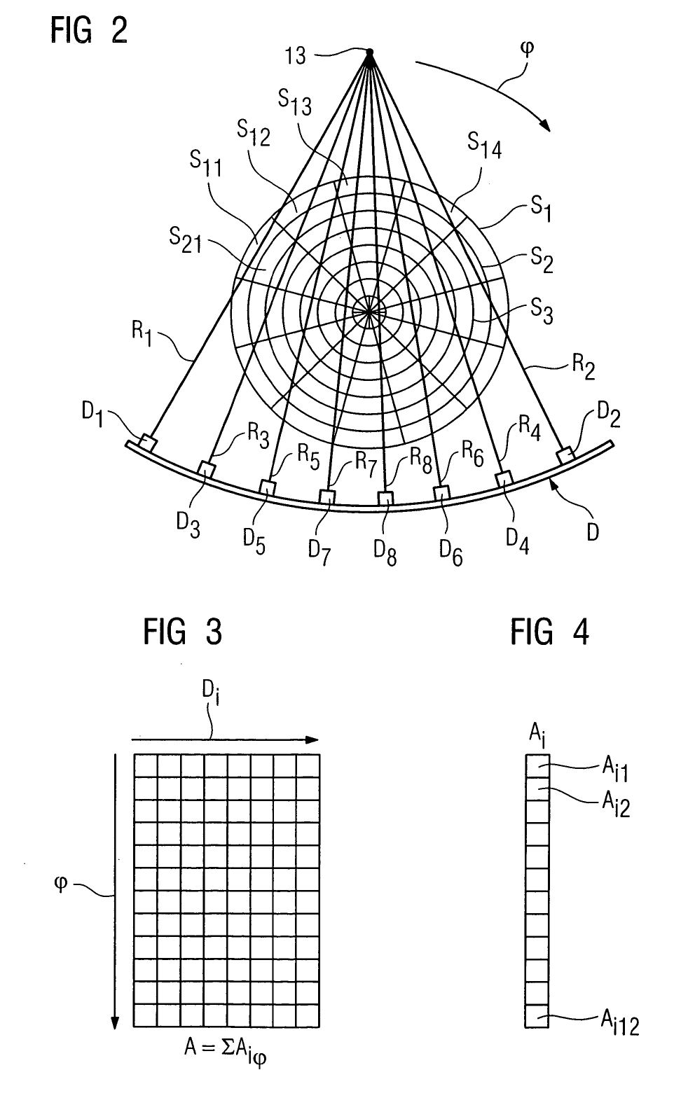

FIGS. 2 to 6 illustrate a special variant of the iterative calculation with shell-type division of the scanning volume.

FIG. 2 shows a focus 13 from which a fan-shaped beam with 8 X-rays R₁ to R₈ strikes an oppositely situated detector D with detector elements D₁ to D₈. During the rotation of the focus 13 and detector about the system axis, each ray R₁ to R₈ scans a shell volume S₁ to S₈ that is divided in turn into 12 shell segments sₛᵢ in accordance with a number of the measured points considered. Thus, each X-ray is assigned a single shell, the detector elements associated with the X-rays being counted according to their distance from the central axis. In the illustration shown, which also corresponds to the detectors that are mostly used, the detector elements are arranged slightly offset from the middle with a so-called “quarter offset” on the detector, such that no redundancies occur between the measured values of the detectors.

During the rotation of the focus 13 and of the detector D about the system axis, each ray R₁ to R₈ scans a shell volume S₁ to S₈ in accordance with its distance from the system axis, the outer ray R₁ penetrating only the outer shell S₁.

It therefore follows that the absorption information of this ray R₁ and of the detector element D₁ suffices for calculating the absorption coefficients of the outer shell. The next ray R₂ lying further inside, exclusively penetrates its shell S₂ and the already calculated shell S₁ lying further outside with reference to their spatial absorption coefficients. It is thereby possible to calculate the absorption coefficients of the shell S₂ in turn solely from the absorption information of the ray R₂ and of the detector element D₂ and the already known absorption coefficients from the shell S₁. This mode of procedure can be continued in accordance with the idea of the invention until the center of the scanning volume is reached and all the absorption coefficients of the subvolumes, here the shell segments, are calculated.

In practice, the results of the scanning, that is to say the detector output values, for each ray and for each detector element and each angle of rotation φ, are plotted so as to obtain a sinogram A as illustrated in FIG. 3, each column corresponding to the measuring points of a detector element, and each row being assigned to the individual measuring points for a 360° revolution.

If a single partial sinogram is considered, that is to say the measured values of an individual detector element for a complete revolution, the result is the partial sinogram as shown in FIG. 4, which here likewise has 12 individual values Aᵢ₁ to Aᵢ₁₂ in accordance with the number of shell segments. Each individual value of this partial sinogram corresponds to the absorption of an X-ray that strikes this considered detector element at the corresponding measuring angle.

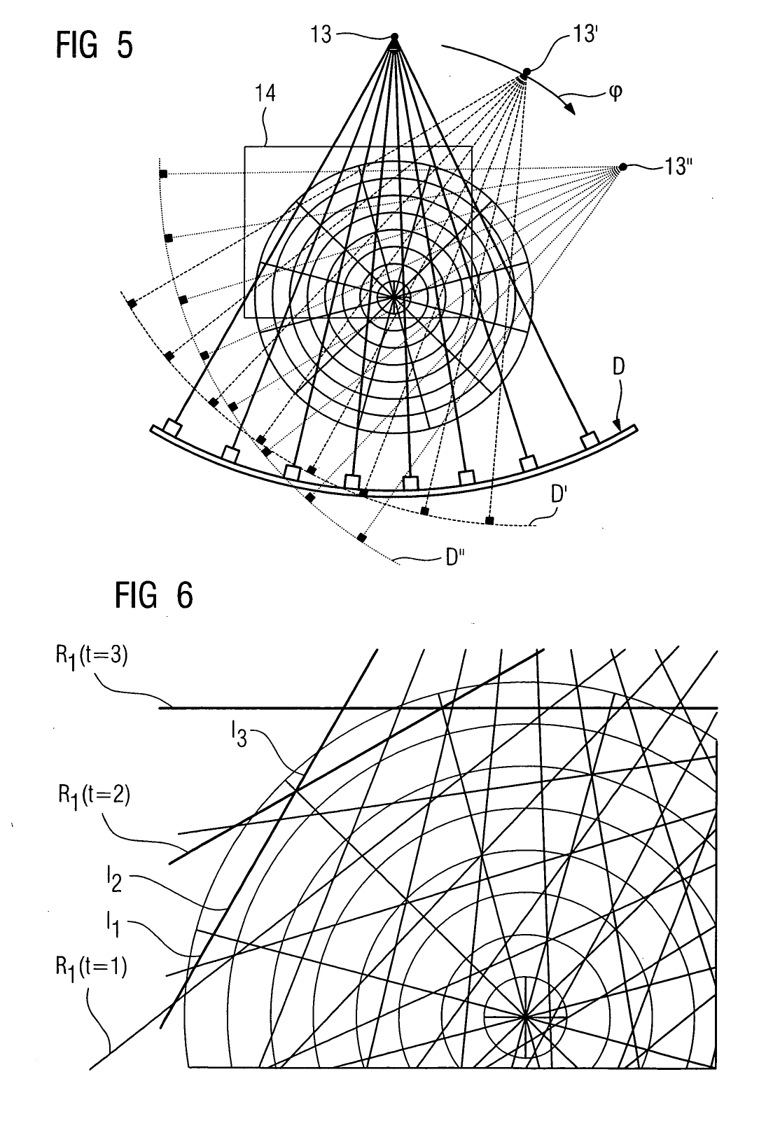

FIG. 5 illustrates the scanning operation of the ray fan for three different angles of rotation φ. The rotated situations are marked by one ′ or two ″. For each partial rotation, the X-rays are displaced so far that a new shell segment is penetrated at their center, further, different shell segments likewise being touched tangentially in the edge regions. It is to be noted, in particular, that in the case of a 360° rotation of the outer X-ray R₁ it is exclusively segments of the outer shell R₁ that are penetrated, and so it is possible to calculate the absorption coefficients of the outer shell without taking account of the values of other detector elements than the detector element D₁, and to carry out this calculation very easily, such that all the absorption coefficients are thereby to hand.

If the next X-ray R situated further inside is considered, it is only the outer shell and the second shell that are penetrated thereby, so that only the measured values of the detector element D₂ and the absorption coefficients, calculated before that, of the shell segments of the first shell are required for calculating the absorption coefficients of the shell segments of the second shell. This way of consideration can be carried out iteratively as far as the inner shell such that a very simple calculation of all the absorption coefficients result therefrom.

In an enlarged illustration of the section 14 from FIG. 5, FIG. 6 shows the paths of the X-rays through the scanning volume subdivided in a shell-type fashion. The X-ray R₁ (t=1, 2 and 3) is emphasized. It is easy to see that the X-ray R₁ (t=1) of length L₂ penetrates the shell segment, assigned to it, on its greatest length, while the lengths l₁ and l₃ relate to the neighboring ray segments. The X-ray R₁ moves through the scanning volume in accordance with the rotation of the focus and detector, it being possible for the effective path lengths with which the individual shell segments are penetrated to be easily calculated.

The division of the scanning volume into individual subvolumes arranged in the shape of shells is illustrated in FIGS. 2 to 6. The iterative calculation is particularly simple because of its geometric association with the scanning method of a cyclically rotating focus. However, the method according to at least one embodiment of the invention is in no way limited to such a shell-type arrangement of scanning volumes, but to the specialist can likewise be transferred to other subdivisions of the scanning volume.

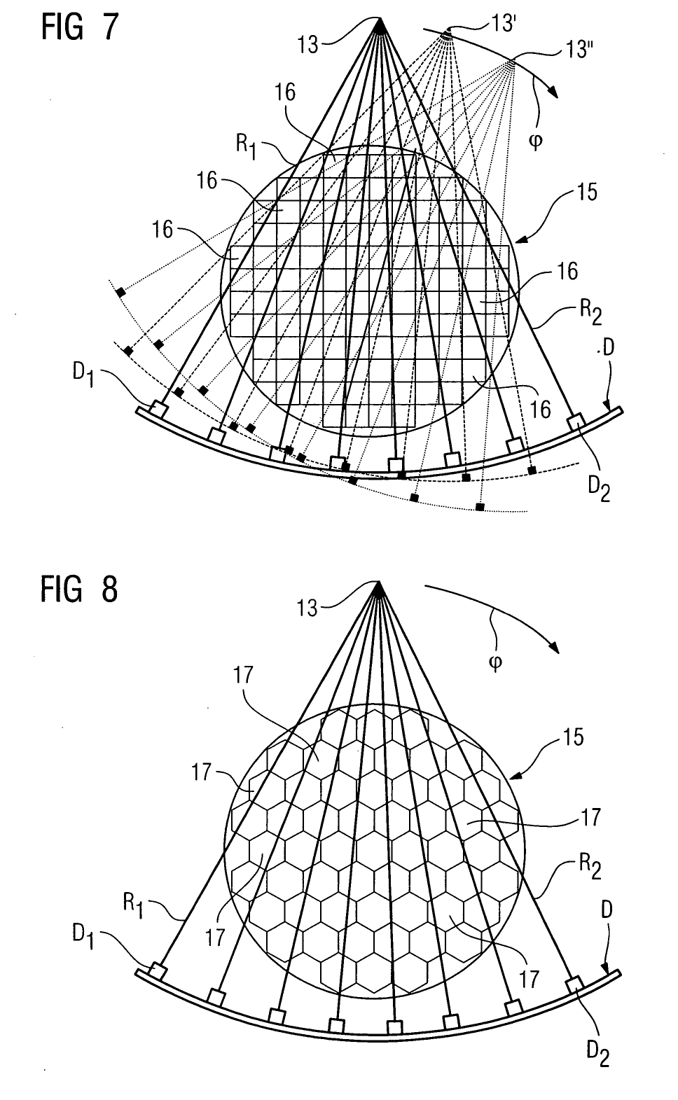

For example, a division of the scanning volume 15 into a multiplicity of subvolumes 16 of square cross section is illustrated in FIG. 7. Moreover, the scanning of the scanning volume by a ray fan emanating from the focus 13 and having X-rays Rₓ in conjunction with different angles of rotation φ is indicated by the addition of ′ and ″. The basic principle of the calculation as outlined above is maintained. All that need be ensured in this case is that the calculation of the absorption coefficients of the subvolumes that are penetrated by an outer first ray are calculated, and thereafter these calculated values are successively taken into account when calculating subvolumes situated further inside and that are penetrated by the X-rays respectively situated next on the inside.

In addition, a honeycomb-shaped division of the scanning volume is shown in FIG. 8, it also being possible to apply the iterative calculating method according to at least one embodiment of the invention for such a division.

The examples outlined so far in each case concern the consideration of a focus/detector combination with a single detector that revolves around an examination object on a circular track. According to at least one embodiment of the invention, it is also possible to apply the method described to such a focus/single-row detector combination that moves spirally, that is to say with synchronous feeding relative to the examination object, such that a spiral track is scanned. An extension of the method to a multirow detector is also possible correspondingly, it being possible for said detector to move both on a circular track and on a spiral track.

The first step for converting the iterative shell reconstruction in 3D is to transfer from a reconstruction in polar coordinates to a reconstruction in cylindrical coordinates. The system axis is thus obtained as third space coordinate. The intersections of the ray lines with the 2D shells result in the intersection with a cylinder.

For multirow CT systems, and in particular, two-dimensional detectors, the fan angle between the central axis of tube and detector and the z position (z-axis=system axis) of a detector row can be above 10 degrees. In the event of static rotation of detector and tube, a plane is no longer defined for a fan angle γ>0. Instead of this, a “diablo-shaped” or “saucer-shaped” volume results for one revolution.

According to at least one embodiment of the invention, the individual rows of the detector are reconstructed iteratively from the outside to the inside taking account of absorption coefficients already previously calculated. The “slice shells” of a detector row, in this case, have an inclination γ to the local mean perpendicular between tube and detector. A diablo-shaped slice volume results from the 360° rotation of the mean perpendicular about the patient axis and is to be manipulated with the shell reconstruction. To this end, the shells are set up for the plane with γ=0. Owing to the fan angle, the absorption lengths of the rays through the shell segments considered rise by a factor 1/cos(γ)>1. Otherwise, the reconstruction is carried out as described above for each revolution for p projections.

Thus, by contrast with the abovedescribed 2D reconstruction method, only a change in absorption lengths that are scaled with a factor 1/cos(γ) results. The method then has the effect of reconstructing a “diablo” instead of a plane. The conditioning of these data in system axis slice planes can now be carried out as follows.

Mean plane approximation: if the aim is to reconstruct planes in the mean plane of the “diablo”, it is then possible to neglect the extended shape for small fan angles: the reason for this is also that contributions of volumes on both sides of the plane are introduced with uniform weighting for the regions of the outer shells that are extended in the z-direction. The method results directly in the reconstruction of nₘₐₓ (=number of detector rows) mean planes. This information can also be interpolated, for example linearly, on to other z-planes.

Radius-dependent Z interpolation: the dependence of the slice resolution on the radius is primarily a property of the CT multirow measuring method. The following is an obvious approach in order, if appropriate, to arrive at an iterative solution for relatively large fan angles and for the 3D spiral: firstly, the mean planes are calculated as described above with the aid of shell reconstruction for each detector row. Subsequently, the resulting “diablo” volumes are imaged on to any desired intermediately situated z-planes, with compensation of the radius-dependent resolution. An aim in this case is for the filtering to achieve a uniformly filtered z-extent in the projected z-plane. The longitudinal extent in the z-direction of the “diablo” is to be considered to this end as a function of the shell radius r with 2r*tan(γ) and the pixel aperture of the detector. As in the case of the 2D method, no lowpass filtering for the purpose of noise reduction is pursued beyond the homogenization of the plane. As a result, maximum information is to be extracted from the raw data even in 3D, and the desired noise/sharpness value is to be incorporated into the display for the first time.

In principle, exactly the same procedure is selected for the shell reconstruction of a spiral scan as with the reconstruction taking account of the fan angle. The volume of a 360 degree revolution defined by the elliptical slice planes is dependent on the magnitude of the CT feed. In the case of very rapid feed, there are two inclined elliptical planes as boundary of the cylindrical slice. The resulting volume looks, so to say, like an obliquely cut slice of sausage. In the case of a small feed, the result returns, by contrast, to the diablo. Finally, mean feed values around 1 produce transitions between the two shapes.

Exactly as when taking account of the fan angle alone, data for a 360° revolution are now reconstructed to form these volumes and subsequently converted as described above to form any desired interpolating z-plane.

Irrespective of the type of scanning, it is advantageous that the slices or subvolumes situated outside, which are scanned exclusively by an outer edge ray, are always firstly calculated solely by way of the measured absorption values of this edge ray, and subsequently the absorption coefficients of slices and subvolumes lying further inside are calculated iteratively, it being permissible to make use here only of rays that intersect subvolumes that either have already been calculated or are penetrated only by this ray. Considering scanning with a multirow detector, then instead of calculating a single trace with the aid of an edge ray, the method according to at least one embodiment of the invention can also begin with the calculation of an entire track of traces that begin from a column of detectors of the multirow detector and their assigned edge rays and are then continued inward iteratively. It can be advantageous here firstly to calculate an entire outer envelope and subsequently to calculate further cylindrical-shell type volumes iteratively in a fashion progressing inward.

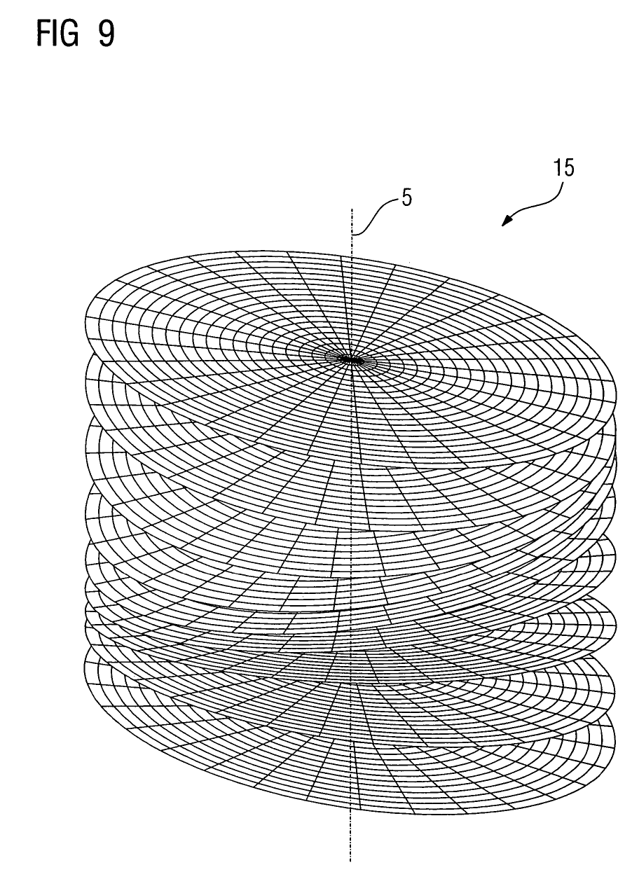

An example 3D scan of a scanning volume 15 and its division into shell segments arranged in the shape of shells is shown in FIG. 9.

Thus, at least one embodiment of the invention proposes overall a reconstruction method in computed tomography in which an examination object is scanned with a beam that rotates about a system axis during scanning, firstly the absorption coefficients of subvolumes that are most remote from the system axis being determined, and subsequently the absorption coefficients of the subvolumes situated closer to the system axis being determined iteratively taking account of the absorption coefficients already calculated. This method can be carried out in each case per se for a single slice plane, or for a number of individual slice planes independently of one another.

However, it can also be carried out in spiral scanning for complete scanning volumes of at least substantially cylindrical design by firstly calculating edge subvolumes by way of edge rays and subsequently iteratively calculating subvolumes approaching the system axis and lying further inside with reference to their absorption coefficients by in each case taking account of the already calculated coefficients lying further outside. This method is also suitable for use with multirow detectors, in which case instead of a ray lying remote from the system axis, consideration is given to a number of rays lying outside in accordance with the row number and to their assigned subvolumes, and then the rays lying further inside and their assigned subvolumes are calculated iteratively. The method according to at least one embodiment of the invention can likewise be used for multifocus or multitube systems.

It is self evident that the abovenamed features of the embodiments of the invention can be used not only in the combination respectively specified, but also in other combinations or on their own without departing from the scope of the invention.

What is claimed is:

1. A reconstruction method in computed tomography, comprising:
scanning an examination object with a beam that rotates during scanning about a system axis;
determining absorption coefficients of subvolumes that are relatively remote from the system axis; and
subsequently determining absorption coefficients of subvolumes relatively nearer to the system axis, iteratively, taking account of the absorption coefficients already calculated.

2. The method as claimed in claim 1, wherein the method is respectively carried out per se for at least one of an individual slice plane and a number of individual slice planes, independently of one another.

3. The method as claimed in claim 1, wherein spiral scanning takes place and it is carried out for complete scanning volumes of at least substantially cylindrical design by firstly calculating subvolumes at the edge using rays at the edge and subsequently iteratively calculating subvolumes, lying relatively further inside and approaching one another in relation to the system axis, with reference to their absorption coefficients, doing so in each case by taking account of the already calculated absorption coefficients lying relatively further outside.

4. The method as claimed in claim 1, wherein at least one of multirow detectors, multifocus systems and multitube systems are used.

5. A method for reconstructing tomographs from detector measured data of a tomography unit, comprising:
moving at least one radiation source about a system axis;
measuring, using at least one oppositely situated at least single row detector, measures absorption of the radiation, emanating from the radiation source after the penetration of an examination object, the radiation source revolving around the examination object on an imaginary cylindrical surface and in the process scanning the examination object, which lies in a scanning volume formed by the rays, with a beam, the scanning volume being divided into a multiplicity of subvolumes;
using a ray in the beam that is remote from the system axis and whose absorption is used to determine absorption coefficients of the subvolumes penetrated by this ray; and
subsequently, iteratively using rays relatively closer to the system axis and using their measured absorption in order to determine as yet unknown absorption coefficients of as yet unconsidered subvolumes, taking account of already known absorption coefficients of already calculated subvolumes.

6. The method as claimed in claim 5, wherein
the scanning volume is divided into a multiplicity of concentric shells, and the shells are subdivided in turn into shell segments, wherein firstly the absorption coefficients of relatively outer segments are determined, and thereupon the absorption coefficients of the shell segments lying relatively further inside, are determined iteratively taking account of the absorption coefficients, calculated meanwhile of shell segments lying relatively further outside, until the center of the scanning volume is reached.

7. The method as claimed in claim 6, wherein the shells are divided into individual shell segments of identical thickness in the radial direction.

8. The method as claimed in claim 6, wherein the shells are divided into individual shell segments of identical length in the circumferential direction.

9. The method as claimed in claim 6, wherein the individual shell segments have an identical cross-sectional surface perpendicular to the system axis.

10. The method as claimed in claim 6, wherein the individual shell segments sweep over a segment angle of identical size.

11. The method as claimed in claim 6, wherein, proceeding from the focus to a specific detector element, each ray is assigned a shell at a constant distance from the system axis.

12. The method as claimed in claim 6, wherein each ray of the beam describes a tangent circle with all the perpendicular feet relative to the system axis, and the shell segments that have an outer circular segment and an inner circular segment, the shell segments being arranged in such a way that the tangent circle lies centrally between the outer circular segment and inner circular segment.

13. The method as claimed in claim 6, wherein each shell segment has an imaginary centroid line that corresponds to a circular segment of concentrically arranged circles about the system axis.

14. The method as claimed in claim 6, wherein each shell segment has an imaginary centroid line that corresponds to a segment of concentrically arranged helical lines about the system axis.

15. The method as claimed in claim 2, wherein the absorption coefficients (\vec{μ}ₛ) of the shell segments is calculated iteratively using the following formula:
$${\vec{μ}}_{s} = {L}_{s}^{- 1} ( {\vec{A}}_{s} - {\sum }_{i = 1}^{s - 1} {L}_{s , i} {\vec{μ}}_{i} ) ,$$
Lₛ being the path length matrix of the defining ray of the shell s, L_{s,i} being the path length matrices of the rays i=1 . . . s lying further outside with reference to the system axis, and Aₛ being the partial sinogram belonging to the shell Sₛ.

16. The method as claimed in claim 1, wherein the subvolumes are of rectangular formation in section perpendicular to the system axis.

17. The method as claimed in claim 1, wherein the subvolumes are of hexagonal formation in section perpendicular to the system axis.

18. The method as claimed in claim 1, wherein, during the iterative measurement of the absorption coefficients, their energy dependence is taken into account.

19. The method as claimed in claim 18, wherein use is made for this purpose of the energy-dependent variation in the intensity of radiation after passage through the examination object.

20. The method as claimed in claim 18, wherein, use is made for this purpose of the total variation in intensity of at least two rays having a known different energy spectrum on the same beam path.

21. The method as claimed in claim 20, wherein at least two radiation sources are used which have different spectra, and which are arranged in such a way that they revolve on the same track during the scanning of the examination object.

22. The method as claimed in claim 1, wherein the examination object is scanned with a beam of known energy spectrum, and the varied energy spectrum of each ray is measured after passage through the examination object.

23. The method as claimed in claim 22, wherein the energy spectrum includes at least two mean energies.

24. The method as claimed in claim 18, wherein an intensity value of a primary color is assigned per energy to the value of the energy-dependent absorption coefficients when displaying the CT tomograms, a color display of the CT image resulting therefrom.

25. A tomography unit for reconstructing tomograms from detector measured data, comprising:
at least one radiation source, movable about a system axis;
at least one oppositely situated, at least single row detector that measures the absorption of the radiation, emanating from the radiation source, after penetration of an examination object, wherein at least the radiation source revolves around the examination object on an imaginary cylindrical surface and scans the examination object, which lies in a scanning volume formed by the rays, with a beam; and
means for determining absorption coefficients of subvolumes that are relatively remote from the system axis and subsequently determining absorption coefficients of subvolumes relatively nearer to the system axis, iteratively, taking account of the absorption coefficients already calculated.

26. The method as claimed in claim 7, wherein the shells are divided into individual shell segments of identical length in the circumferential direction.

27. The tomography unit as claimed in claim 25, further comprising:
means for controlling the tomography unit and for collecting and computationally processing detector output data,
reconstructing tomographic images and displaying the images.
